(19) United States
(12) Patent Application Publication
(10) Pub. No.: US 2023/0110299 A1
(43) Pub. Date: Apr. 13, 2023

(54) Hygiene Base Composition, and Production Method and Application Method Thereof

(71) Applicant: Qifeng MA, Rancho Palos Verdes, CA (US)

(72) Inventor: Qifeng MA, Rancho Palos Verdes, CA (US)

(21) Appl. No.: 17/496,993

(22) Filed: Oct. 8, 2021

(51) Int. Cl. A61K 8/9789; A61K 8/33

(52) U.S. Cl. CPC A61K 8/9789; A61K 8/33; A61K 2800/10

(57) ABSTRACT

A hygiene base composition includes a composition base which includes a cinnamon extract, and a carrier, wherein the composition base is carried by the carrier to form a hygiene product for applying on a defined target. The hygiene base composition has the features of anti-bacteria, anti-fungi, and anti-virus activity. The hygiene product, containing the cinnamon extract, is configured as one of an oral hygiene product, a skin hygiene product, a breath tract hygiene product, an atmosphere hygiene product, a wound hygiene product, an athlete foot hygiene product, an insect bite hygiene product, an acne hygiene product, a tooth hygiene product, an oral ulcer hygiene product, and a throat hygiene product.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates to hygiene composition, and more particularly to a hygiene base composition, and preparation method and application method thereof.

### Description of Related Arts

Cinnamon has been used in traditional oriental medicine for thousands of years. It is described as “warming stomach and kidney, relieving stomach pain, ill digestion, diarrhea, and waist pain”. In the western countries, cinnamon is widely used as cooking spice and food or beverage ingredient. Modern research found that cinnamon contains many volatile natural compounds that produce the special cinnamon flavor and many activities such as anti-microbial property. Cinnamaldehyde is one of them. Previous research found Cinnamaldehyde has anti-bacteria, anti-fungi, and anti-virus activity (Carlos R. Polaquini, et al. 2017). Unfortunately, cinnamaldehyde has very short half-life when it is delivered into human body, preventing its further development in the health care industry.

Hygiene products nowadays are all synthetic compounds and have various side effects. Not all anti-microbial compounds can be a hygiene reagent due to many factors such as toxicity. Anti-biotic drugs are less toxic. Since it will result in drug-resistant bacteria when they are used for hygiene, they cannot become hygiene ingredients. It is well-known that natural products are good to human beings. If a hygiene formulation can be developed from natural products, it would be advantageous and beneficial to the health care field.

### SUMMARY OF THE PRESENT INVENTION

The invention is advantageous in that it provides a hygiene base composition, and preparation method and application method thereof, wherein the hygiene base composition which is a combination of cinnamon extract and a carrier is used in the health care field for hygiene of a defined target. Taking advantage of its mild volatile property, the cinnamon containing formulation can have more applications than the existing hygiene products.

Another advantage of the invention is to provide a hygiene base composition, which is natural, edible and non-toxic to human being, such that the hygiene base composition is safe to use.

Another advantage of the invention is to provide a hygiene base composition, which can be used as an oral hygiene product, a skin hygiene product, a breath tract hygiene product, an atmosphere hygiene product, a wound hygiene product, an athlete foot hygiene product, an insect bite hygiene product, an acne hygiene product, a tooth hygiene product, an oral ulcer hygiene product, and/or a throat hygiene product.

Another advantage of the invention is to provide a hygiene base composition, which has the features of anti-bacteria, anti-fungi and anti-virus activity. Furthermore, the hygiene base composition has no side effect after being used and has relatively long effective life comparing to the existing hygiene products.

Another advantage of the invention is to provide a hygiene base composition and preparation method thereof, wherein the application of the hygiene base composition is easy by simply applying the hygiene base composition to the target.

Another advantage of the invention is to provide a hygiene base composition, which does not involve complicated and expensive components and processes so that the manufacturing cost of the present invention can be minimized.

Another object of the present invention is to provide a hygiene base composition, wherein no expensive and complicated structure and configuration is required to be employed in order to achieve the above mentioned advantages and objects of the present invention. Therefore, the present invention successfully provides an economic and efficient solution for applying the cinnamon extract in various applications.

Additional advantages and features of the invention will become apparent from the description which follows and may be realized by means of the instrumentalities and combinations particular point out in the appended claims.

According to the present invention, the foregoing and other objects and advantages are attained by a hygiene base composition, comprising:
a composition base which comprises a cinnamon extract; and
a carrier, wherein the composition base is carried by the carrier to form a hygiene product for applying on a defined target.

In accordance with another aspect of the invention, the present invention comprises a preparation method of a hygiene base composition, comprising the following steps.

(A) Form a composition base which comprises at least a cinnamon extract.

(B) Provide a carrier that the composition base is carried by the carrier to form a hygiene product for applying on a defined target.

In one embodiment, the step (A) of the preparation method of the present invention comprises a step of dissolving a predetermined weight of the cinnamon extract in a volatile solvent such as ethanol, wherein the amount of ethanol to be used should be enough to fully dissolve the cinnamon extract in the concentration from 0.1% to 50% to form the composition base in liquid form for well mixing with 80 to 120 times (preferably 100 times) of the predetermined weight of the cinnamon extract of the carrier in the step (B). Thereafter, the ethanol solvent is preferred to be evaporated to form the hygiene base composition.

The carrier can be a porous silica in solid form or a water in liquid form according to various alternative modes of the preferred embodiment of the present invention.

In one embodiment, a solid form carrier is used, which can be embodied as a porous sheet, wherein the composition base is applied to the surface of the porous sheet.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a block diagram illustrating a hygiene base composition according to a first preferred embodiment of the present invention.

FIG. 2 is a block diagram illustrating a hygiene base composition according to a second preferred embodiment of the present invention.

FIG. 3 is a block diagram illustrating a hygiene base composition according to a third preferred embodiment of the present invention.

FIG. 4 is a flow chart illustrating a production method of the hygiene base composition according to the first to third preferred embodiments of the present invention.

FIG. 5 is a table showing microorganism inhibition by cinnamon extract of different concentration according to the first to third preferred embodiments of the present invention.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

Referring to FIG. 1 of the drawings, a hygiene base composition, according to a first preferred embodiment of the present invention, comprises a composition base and a carrier carrying the composition base. According to the first preferred embodiment, the carrier has a porous solid physical form, such as powders, granules, and sheet form. The composition base comprises a cinnamon extract dissolved in an organic volatile solvent, preferably ethanol, in the concentration from 0.1% to 50% to form a solution. The solution is adapted to spread onto the surface of the carrier and then the organic solvent is removed from the solution to deposit the cinnamon extract onto the carrier to form the hygiene base composition.

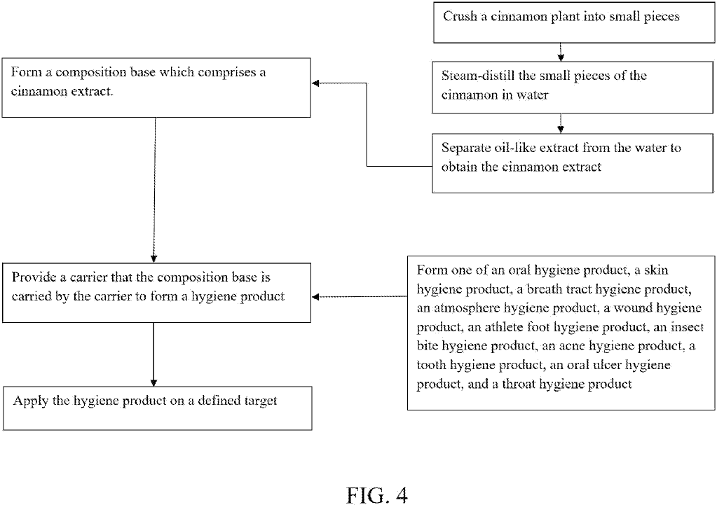

Referring to FIG. 4, a preparation method of the hygiene base composition comprises the following steps.

(A) Form a composition base which comprises at least a cinnamon extract.

(B) Provide a carrier that the composition base is carried by the carrier to form a hygiene base composition for applying on a defined target.

Accordingly, the step (A) of the present invention further includes a preparation method of the cinnamon extract, including:
crushing 1 kg of cinnamon plant into small pieces which are steam-distilled to collect 10 kg of distilled water; and
separating oil-like extract from water by keeping the distilled water at 10° C. overnight until 9.5 ml of the cinnamon extract is obtained, wherein cinnamaldehyde concentration in the cinnamon extract is 76% by HPLC analysis.

Accordingly, the preparation method of the hygiene base composition according to the first preferred embodiment with porous powder as the carrier includes the steps of:
(a) dissolving 0.1 gram of cinnamon extract in 5 ml of ethanol to form the composition base in the concentration from 0.1% to 50%;
(b) mixing the composition base with 10 gram of porous silica as the carrier; and
(c) evaporating away the ethanol from the carrier (the porous silica) to form the hygiene base composition.

According to the first preferred embodiment, the preparation method further comprises a step (d) of retaining the hygiene base composition in a sealed aluminum bag for use.

In the step (a), the 0.1 gram of cinnamon extract used is merely an example. Also, the 5 ml of ethanol to dissolve the cinnamon extract is an example accordingly to form the composition base. In the step (b), the 80-5000 times (preferably 1000 times) of the weight of the cinnamon extract of porous silica is used as the carrier to be mixed with the solution form composition base. In other words, the proportion of the composition base and the carrier is 1:80-5000, preferably 1:1000.

Alternatively, the carrier in the step (b) of the preparation method of hygiene base composition according to the first preferred embodiment is a porous sheet such as a three-layer cheese cloth sheet, wherein 0.1 gram of the cinnamon extract dissolved in 10 ml of ethanol to form a solution form of the composition base which is further evenly sprayed onto the three-layer cheese cloth sheet of 10 cm by 80 cm (width by length). Therefore, in the step (c), the ethanol is then evaporated away from the cheese cloth sheet to form the hygiene base composition. Also, the hygiene base composition is kept in a sealed aluminum bag for use.

The hygiene base composition according to the first preferred embodiment can be used for atmosphere hygiene in a closed environment such as in a building, a vehicle, an airplane, a ship, a refrigerator, or a cabinet. The carrier of the first preferred embodiment can be placed in the desired environment to allow the molecules of the cinnamon extract evaporating into the atmosphere. Heating of the carrier can enhance the strength of hygiene. The atmosphere in the closed environment can also circulate through the carrier to blow the molecules of cinnamon extract into the closed environment, enhancing the strength of atmosphere hygiene.

The hygiene base composition of the first embodiment can also be used for hygiene of human breathing tract. In one specific application, the porous solid carrier can be porous sheet made, for example, in form of a mouth mask, wherein the composition base of the first preferred embodiment is applied on the mask carrier. The molecules of the cinnamon extract are carried into breathing tract by breath fresh air through the carrier mask and functions for hygiene of breathing tract. Likewise, the porous sheet can be embodied as an air filter as the carrier, wherein the composition base of the first preferred embodiment is applied on the air filter.

The composition base of the first preferred embodiment is a base composition for producing a hygiene composition product, wherein the composition base can combine with other ingredients to form various hygiene composition products for different applications. In the case of hygiene in a closed environment, volatile air refreshing ingredients and/or deodorants can be combined with the composition base and be carried by the carrier. In the case of hygiene of human breathing tract, volatile sweetener, anti-inflammation reagents, hygiene-effect-enhancing ingredients, and/or breathing tract symptom relief ingredients can be combined with the composition base and be carried by the carrier. Many other ingredients can be combined to the composition base of the first preferred embodiment and the examples above are for exemplary only and are not limited in the present invention.

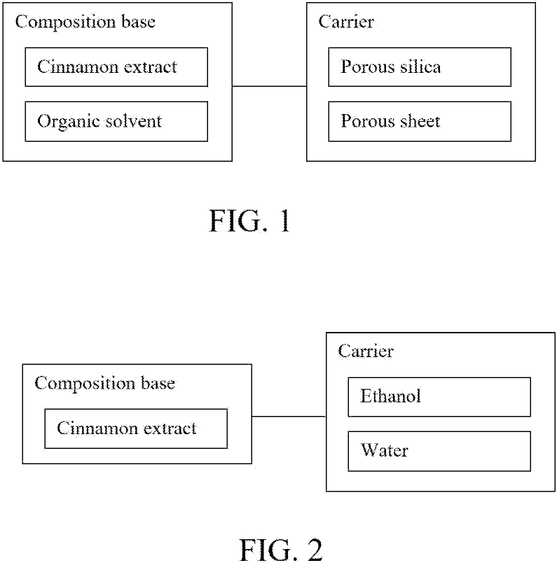

As shown in FIG. 2, a second preferred embodiment of the present invention illustrates an alternative mode of the carrier of the present invention, wherein the carrier is in form of volatile liquid to carry the composition base. The volatile liquid to be used as the carrier to carry, i.e. to retain, the composition base of the present invention should be non-toxic to human body and has a volatility higher than the volatility of the cinnamon extract of the composition base. In other words, the volatile liquid carrier should be more volatile than the cinnamon extract and should be non-toxic to the human being. The volatile liquid carrier functions to carry the molecules of cinnamon extract to the hygiene target, evaporating away and leave the cinnamon extract onto the target. In practice, cinnamon extract is dissolved in the volatile liquid carrier to form the hygiene base composition according to the second preferred embodiment.

Accordingly, the preparation of the hygiene base composition of the second embodiment with ethanol as the carrier is that 0.1 gram of cinnamon extract is dissolved in 100 ml of ethanol. The hygiene base composition is kept in a sealed glass bottle for use.

Alternatively, the preparation method of the hygiene base composition according to the second preferred embodiment with water as the carrier includes the steps of:
(a) providing 0.1 gram of the cinnamon extract as the composition base;
(b) dissolving the composition base in 100 ml of ethanol and then mixing with 10 gram of water as the carrier of the composition base; and
(c) evaporating away the ethanol to form the hygiene base composition.

The hygiene base composition prepared by the second preferred embodiment of the present invention is preferred to be kept in a sealed glass bottle for use.

According to the second preferred embodiment, the step (c) can be omitted or is preferred to not completely evaporate the ethanol so that the carrier will become a volatile carrier of the composition base that facilitates the application of the hygiene base composition to be applied on an object surface. After the ethanol content in the volatile carrier is evaporated, the cinnamon extract is able to be well retained on the object surface.

In other words, the hygiene base composition according to the second preferred embodiment is used for hygiene of a surface. The surface includes surface of an object and certain human body surfaces such as skin and mouth. In the case of object surface hygiene, the hygiene base composition can be spread to the object surface by any conventional means that include wipe and spray. The hygiene base composition is configured to firstly deposit onto the object surface, and the volatile carrier will then evaporate away to leave the molecules of cinnamon extract onto the object surface for sustaining hygiene.

In the case of hygiene of the surface of certain human body surface, the hygiene base composition can be spread to the defined body surface by any conventional means that include wipe, spray, and mist inhalation. The hygiene base composition is configured to firstly deposit onto the external or internal body surface, the volatile carrier will then evaporate away to leave the molecules of cinnamon extract onto the body surface for sustaining hygiene. The body surface hygiene can be used for prevention and treatment of some diseases. Hygiene of hands can prevent contact transmission of infectious diseases. Hygiene of breathing tract can kill the microorganisms adhere on the surface of the tract, preventing the air-bore transmission of infectious diseases. Hygiene of skin can not only cure certain topical diseases caused by microorganism infection as of athlete foot, but also prevent possible microorganism complexion of certain non-infectious topical diseases as of wound and acne. Hygiene of oral cavity can treat and prevent of gum, throat, tooth and tongue diseases caused by micro-organism infection.

The liquid carrier should satisfy at least the two key requirements-higher volatile than cinnamon extract and not toxic to human being. Water, ethanol, and hexane are among them. Choice of the liquid as carrier varies case by case. For hygiene of object surface, water, ethanol, and hexane are suitable carrier. For hygiene of skin and hands, both water and ethanol can be used as the carrier. For hygiene of sensitive organ surface such as breathing tract and mouth, water should be used as the carrier.

The composition base according to the second preferred embodiment is capable of further combining other ingredients for different applications. For example, in the case of skin hygiene the composition base can combine with skin care ingredients. In the case of oral hygiene the composition base can combine with air refreshing ingredients. Various ingredients can be combined to the composition base and the examples above are for exemplary only and are not limited in the present invention.

The hygiene base composition according to the present invention has at least the following features that distinguish from the conventional art. The composition base is a natural food in nature, edible and not toxic. The molecules of cinnamon extract in the hygiene base composition are volatile and hygiene the atmosphere of a closed environment. The molecules of the active components are not too volatile that it can stay on the surface for sustainable hygiene. All these features differ from the conventional art of hygiene products one way or the other. For example, 75% ethanol pad is the most widely used hand hygiene product, which can only kill microorganisms at 75% ethanol concentration and loss effect immediately after application due to ethanol evaporation, resulting in no sustainable hygiene effect. It has side effects such as irritation and cannot used for human organ besides skin. It also no effect for atmosphere hygiene. The environment hygiene agent hypochlorite is toxic and cannot be used for personal hygiene. Even for environment hygiene, hypochlorite cannot kill germs in the atmosphere since it is not volatile. The present invention substantially provides a method of preparing a composition base from the cinnamon extract as well as a way to form the hygiene base composition from the cinnamon extract through the carrier such that the hygiene base composition can be applied to a closed environment or an object surface.

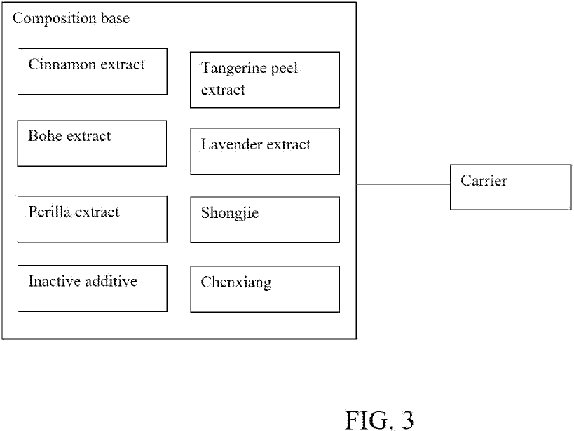

As shown in FIG. 3, a third preferred embodiment illustrates another alternative mode of the hygiene base composition according to the present invention, wherein the composition base provided in the step (A) of the preparation method of the present invention comprises mint extract, cinnamon extract, perilla extract, tangerine peel extract, lavender extract, pine extract, agilawood extract, and an inactive additive. The weight ratio is that mint extract 0-30 parts, cinnamon extract 30-100 parts, perilla extract 0-20 parts, tangerine peel extract 0-30 parts, lavender extract 0-15 parts, pine extract 0-15 parts, agilawood extract 0-12 parts. The inactive additive comprises one or more of water, ethanol, starch, sweetener, and fragrance. Accordingly, the hygiene base composition is preferred to be used for personal hygiene and atmosphere hygiene in a closed environment, such as in a building, a vehicle, a bathroom, a refrigerator, a kitchen cabinet, and a toilet. The personal hygiene includes oral hygiene, skin hygiene, and breathing tract hygiene. The extracts are preferably prepared by steam distillation. The cinnamon extract is the cinnamon element extracted from branch, leaf, and bark of the plant, wherein the cinnamon extract contains cinnamaldehyde compound in a range between 30% to 100%.

According to the above embodiments, the hygiene base composition can be used in different applications such as an oral hygiene product, a skin hygiene product, a breath tract hygiene product, an atmosphere hygiene product, a wound hygiene product, an athlete foot hygiene product, an insect bite hygiene product, an acne hygiene product, a tooth hygiene product, an oral ulcer hygiene product, and a throat hygiene product.

The hygiene base composition can be used for oral hygiene which is performed by delivering the carrier into mouth, wherein the carrier is embodied as mouth wash liquid or mouth liquid spray. The hygiene base composition as the oral hygiene product is configured for disinfection for oral surgery and for relieving pain, swell, and inflammation caused by gum disease, bad tooth, sore throat, and mouth ulcer.

The hygiene base composition can be used for skin hygiene which is performed by delivering the carrier onto skin, wherein the carrier is embodied as topical massage liquid or topical liquid spray. The hygiene base composition as the skin hygiene is configured for disinfecting skin and for relieving itch, swell, and inflammation caused by skin allergy eczema, skin rash, insect bite, acne, athlete’s foot, and skin wound. The hygiene base composition can be further made into hand wash liquid and soap for hand hygiene that kills germs and prevents transmission of infectious diseases.

The hygiene base composition can be used for breath tract hygiene which is performed by delivering the carrier into the breath tract that include nose, throat, and respiratory tract, wherein the carrier can be, but not limited to, liquid mist and liquid spray that are delivered into breath tract through breathing. The hygiene base composition as the breath tract hygiene product is configured for disinfecting the breath tract to kill any microorganism in the tract and to further prevent respiratory transmitted infectious diseases and for relieving inflammation symptoms caused by breath tract infection.

The hygiene base composition can be used for atmosphere hygiene in a closed environment which is performed by spreading the carrier in the closed environment, wherein the carrier can be volatile air refresher and liquid spray. The hygiene base composition as the atmosphere hygiene product is configured for killing germs in the enclosed environment and further for prevention of airborne infectious diseases, for refreshing air, and for removal of bad smell.

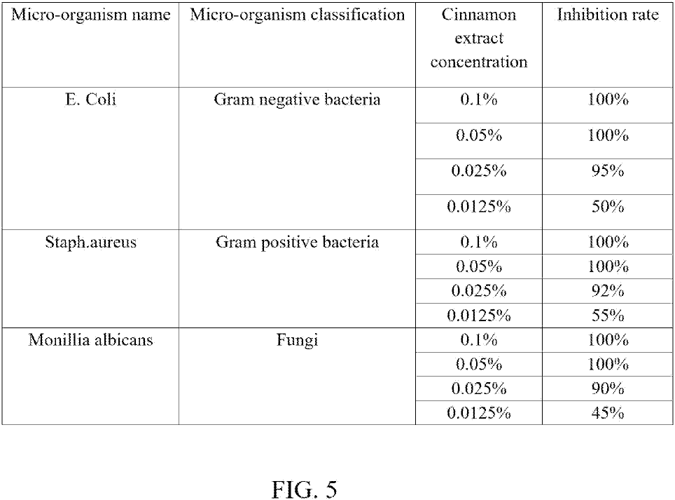

The advantages of the present invention are proven through an anti-microbial test of the cinnamon extract. Cinnamon extract is mixed with water to form a series of cinnamon extract solutions containing 0.1%, 0.05%, 0.025%, 0.0125% and 0.00% cinnamon extract respectively. 150 ml of each solution is then mixed with 1.5 g of protein peptide, 0.75 g of yeast extract, 0.15 g of sodium chloride, and 1.05 g of agar. The mixture is autoclaved and poured into 3 culture dishes to form culture plate. 0.5 ml of bacteria culture is evenly laid on the culture plate and incubated at 37° C. for 24 hours and the bacteria colony number were counted and averaged for each cinnamon extract concentration. The culture plate with 0.0% cinnamon extract is used as a control and the inhibition rate of a defined cinnamon extract concentration is calculated by the following equation:

$${\text{I}}_{\text{x}} = \left(1 - {\text{N}}_{\text{x}} / {\text{N}}_{0}\right) \text{x100\%}$$

wherein Iₓ is inhibition rate of x concentration of cinnamon extract, Nₓ is the colony number of the culture plate of x concentration of cinnamon extract, and N₀ is the colony number of the culture plate without cinnamon extract. FIG. 5 is a table showing microorganism inhibition by cinnamon extract of different concentration.

In view of the application method of the hygiene base composition of the present invention, in one example of the atmosphere hygiene for a bedroom of 20 square meters, the hygiene base composition according to the first preferred embodiment with silica powder as the carrier is placed on a hot plate with surface temperature at 50° C. and is kept there for atmosphere hygiene and deodorization. The components of cinnamon extract evaporated and were detected after 5 minutes in the air of the bedroom.

In another example of the in-car atmosphere hygiene, the hygiene base composition according to the first preferred embodiment with cheese cloth sheet as the carrier, wherein the cheese cloth sheet with the composition base is cut into a sheet of 10 cm by 8 cm in size and is used to cover one ventilator. The car ventilation is set to in-car air circulation mode and then press the start button. The components of cinnamon extract are detected after 2 minutes and the odor in the car is eliminated after 10 minutes of air circulation.

In one example of the breathing tract hygiene, the hygiene base composition according to the first preferred embodiment with cheese cloth sheet as the carrier, the cheese cloth sheet applied with the composition base is cut into a sheet of 10 cm by 8 cm in size and is clipped to the mouth mask facing the mouth. The components of cinnamon extract are carried by the breathing air and travel through the breath tract for hygiene. It is used for killing microorganisms in the breathing tract, resulting in prevention of transmission of air-borne infectious diseases.

In another example of the breathing tract hygiene, the hygiene base composition according to the second preferred embodiment with water as the carrier is placed in a mist generating bottle and placed by nose for duration of 5 minutes or longer. The mist containing the components of cinnamon extract is breathed through the breathing tract for killing microorganisms in the breathing tract, resulting in prevention of transmission of air-borne infectious diseases.

In one example of the wound hygiene, the hygiene base composition according to the second preferred embodiment with water as the carrier is used to wash the minor skin wound and functions for wound hygiene as well as stop bleeding.

In one example of the athlete foot hygiene, the hygiene base composition according to the second preferred embodiment with ethanol as the carrier is filled into a spray bottle and sprayed to the athlete foot three times a day. It functions for killing germs, eliminating odor, and stopping itch.

In one example of the skin hygiene, the hygiene base composition according to the second embodiment with water as the carrier is spread to the target skin site for skin hygiene as desired. The target sites include acne, insect bite, and hands. Hand hygiene also functions for prevention of contact transmission of infectious diseases.

In one example of the oral hygiene, a desired volume of the hygiene base composition according to the second preferred embodiment with water as the carrier is sipped into mouth and kept in the mouth for a while for oral hygiene. It is used for oral ulcer, bad breathing smell, tooth-ache, loosing tooth, gum diseases and other symptoms caused by oral infection.

Another example of the oral hygiene is that the hygiene base composition according to the third preferred embodiment with water as the carrier includes the defined composition base which contains 30% (0.03 gram) mint extract and 70% of cinnamon extract (0.07 gram) and 0% (0.00 gram) of the rest ingredients. The composition base was mixed with 100 ml of water (carrier) to form the hygiene base composition. A desired volume of the hygiene base composition is sipped into mouth and kept in the mouth for a while for oral hygiene. It is used for oral ulcer, bad breathing smell, tooth-ache, loosing tooth, gum diseases and other symptoms caused by oral infection.

The hygiene base composition produced according to the above preferred embodiments is prepared for applying on the defined target preferably by directly spreading to the defined target, wherein the defined target is selected from a group consisting of oral hygiene, skin hygiene, breath tract hygiene, atmosphere hygiene, skin wound hygiene, athlete foot hygiene, insect bite hygiene, acne hygiene, tooth hygiene, oral ulcer hygiene, and a throat hygiene.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A hygiene base composition, comprising:
a composition base which comprises at least a cinnamon extract containing cinnamaldehyde in a range between 30% to 100%; and
a carrier mixed with said composition base in a proportion 1:80-5000, such that said composition base is carried by said carrier to form a hygiene product for applying on a defined target.

2. The hygiene base composition, as recited in claim 1, wherein said carrier is selected from a group consisting of porous silica, porous sheet, ethanol, water, and a combination thereof, and said carrier is mixed with said composition base in a proportion 1:80-5000.

3. The hygiene base composition, as recited in claim 1, wherein said composition base further comprises one or more of a mint extract, a perilla extract, a tangerine peel extract, a lavender extract, a pine extract, an agilawood extract, and an inactive additive mixed with said cinnamon extract.

4. The hygiene base composition, as recited in claim 2, wherein said composition base further comprises one or more of a mint extract, a perilla extract, a tangerine peel extract, a lavender extract, a pine extract, an agilawood extract, and an inactive additive mixed with said cinnamon extract.

5. The hygiene base composition, as recited in claim 3, wherein a weight ratio of said composition base is that said mint extract 0-30 parts, said cinnamon extract 30-100 parts, said perilla extract 0-20 parts, said tangerine peel extract 0-30 parts, said lavender 0-15 parts, said pine extract 0-15 parts, and said agilawood extract 0-12 parts.

6. The hygiene base composition, as recited in claim 4, wherein a weight ratio of said composition base is that said mint extract 0-30 parts, said cinnamon extract 30-100 parts, said perilla extract 0-20 parts, said tangerine peel extract 0-30 parts, said lavender 0-15 parts, said pine extract 0-15 parts, and said agilawood extract 0-12 parts.

7. A preparation method of a hygiene base composition, comprising steps of:
(A) forming a composition base which comprises at least a cinnamon extract in a range between 30% to 100%; and
(B) providing a carrier for mixing with said composition base to form a hygiene base composition for applying on a defined target.

8. The preparation method, as recited in claim 1, wherein the step (A) comprises a step of dissolving a predetermined weight of said cinnamon extract in a volatile solvent to fully dissolve said cinnamon extract in a concentration from 0.1% to 50% to form said composition base in liquid form, wherein in the step (B), said composition base in liquid form is mixed with 80 to 5000 times of said carrier, wherein after the step (B), the preparation method further comprises a step of evaporating said volatile solvent to form the hygiene base composition.

9. The hygiene base composition, as recited in claim 8, wherein said cinnamon extract in said composition base is prepared by the following steps:
(i) crushing a predetermined weight of cinnamon plants of cinnamon into small pieces;
(ii) steam-distilling said small pieces of said cinnamon plant in water to collect a predetermined weight of distilled water; and
(iii) separating oil-like extract from said distilled water by keeping said distilled water at a predetermined temperature for a predetermined period of time to obtain said cinnamon extract to form said composition base.

10. The preparation method, as recited in claim 8, wherein said carrier is selected from a group consisting of porous silica, porous sheet, ethanol, water, and a combination thereof.

11. The preparation method, as recited in claim 7, further comprising steps of:
(a) after the step (A), dissolving said cinnamon extract in a predetermined amount of an organic solvent to form a solution of said composition base;
(b) after the step (B), absorbing said solution of said composition base to said carrier by mixing said solution of said composition base with said carrier; and
(c) evaporating away said organic solvent to from said hygiene base composition.

12. The preparation method, as recited in claim 11, wherein said composition base further comprises one or more of a mint extract, a perilla extract, a tangerine peel extract, a lavender extract, a pine extract, an agilawood extract, and an inactive additive mixed with said cinnamon extract.

13. The preparation method, as recited in claim 12, wherein a weight ratio of said composition base is that said mint extract 0-30 parts, said cinnamon extract 30-100 parts, said perilla extract 0-20 parts, said tangerine peel extract 0-30 parts, said lavender 0-15 parts, said pine extract 0-15 parts, and said agilawood extract 0-12 parts.

14. The preparation method, as recited in claim 11, wherein said organic solvent is an ethanol and said carrier is a porous silica in granule form, wherein said hygiene base composition in granule form is adapted to be placed in a closed environment to allow molecules of said composition base to evaporate into the space for atmosphere hygiene.

15. The preparation method, as recited in claim 13, wherein said organic solvent is an ethanol and said carrier is a porous silica in granule form, wherein said hygiene base composition in granule form is adapted to be placed in a closed environment to allow molecules of said composition base to evaporate into the space for atmosphere hygiene.

16. The preparation method, as recited in claim 11, wherein said organic solvent is an ethanol and said carrier is a porous sheet, wherein said hygiene base composition in sheet form is adapted to be placed in an air circulation path in a closed environment to allow molecules of said composition base to evaporate into a space for atmosphere hygiene.

17. The preparation method, as recited in claim 13, wherein said organic solvent is an ethanol and said carrier is a porous sheet, wherein said hygiene base composition in sheet form is adapted to be placed in an air circulation path in a closed environment to allow molecules of said composition base to evaporate into a space for atmosphere hygiene.

18. The preparation method, as recited in claim 11, wherein said organic solvent is an ethanol and said carrier is a porous sheet, wherein said hygiene base composition in sheet form is adapted to be placed in a breathing path of a person to allow molecules of said composition base to flow through a breathing tract for breathing tract hygiene.

19. The preparation method, as recited in claim 13, wherein said organic solvent is an ethanol and said carrier is a porous sheet, wherein said hygiene base composition in sheet form is adapted to be placed in a breathing path of a person to allow molecules of said composition base to flow through a breathing tract for breathing tract hygiene.

20. The preparation method, as recited in claim 11, wherein said carrier is in form of volatile liquid to carry said composition base to form said hygiene base composition in volatile liquid form adapted for applying on an object surface, wherein a volatility of said carrier in volatile liquid form is higher than a volatility of said composition base.

21. The preparation method, as recited in claim 20, wherein in the step (b), said carrier comprising a predetermined amount of said ethanol mixed with a predetermine amount of said water.

22. The hygiene base composition, as recited in claim 8, wherein said hygiene base composition is directly spread to the defined target, wherein said defined target is selected from a group consisting of oral hygiene, skin hygiene, breath tract hygiene, atmosphere hygiene, skin wound hygiene, athlete foot hygiene, insect bite hygiene, acne hygiene, tooth hygiene, oral ulcer hygiene, and a throat hygiene.

23. The hygiene base composition, as recited in claim 15, wherein said hygiene base composition is directly spread to the defined target, wherein said defined target is selected from a group consisting of oral hygiene, skin hygiene, breath tract hygiene, atmosphere hygiene, skin wound hygiene, athlete foot hygiene, insect bite hygiene, acne hygiene, tooth hygiene, oral ulcer hygiene, and a throat hygiene.

24. The hygiene base composition, as recited in claim 17, wherein said hygiene base composition is directly spread to the defined target, wherein said defined target is selected from a group consisting of oral hygiene, skin hygiene, breath tract hygiene, atmosphere hygiene, skin wound hygiene, athlete foot hygiene, insect bite hygiene, acne hygiene, tooth hygiene, oral ulcer hygiene, and a throat hygiene.

25. The hygiene base composition, as recited in claim 19, wherein said hygiene base composition is directly spread to the defined target, wherein said defined target is selected from a group consisting of oral hygiene, skin hygiene, breath tract hygiene, atmosphere hygiene, skin wound hygiene, athlete foot hygiene, insect bite hygiene, acne hygiene, tooth hygiene, oral ulcer hygiene, and a throat hygiene.
